(19) United States
(12) Patent Application Publication
(10) Pub. No.: US 2011/0017856 A1
(43) Pub. Date: Jan. 27, 2011

(54) Retractable (medical) oxygen tubing reel

(76) Inventor: Amy J. Penn, Sarver, PA (US)

Correspondence Address: Amy J. Penn, 679 Sarver Rd, Sarver, PA, 16055, US

(21) Appl. No.: 12/379,710

(22) Filed: Jul. 22, 2009

(51) Int. Cl. A61M 39/08; B65H 75/34; B65H 75/44; B65H 75/40

(52) U.S. Cl. 242/370

(57) ABSTRACT

An apparatus to aid in the holding and retention of oxygen tubing used in a health care environment comprising approximately fifty (50) feet of oxygen tubing wound upon a retractable reel is herein disclosed. A spring-loaded tensioning system is provided for automatic retraction of the oxygen line. The spring-loaded system would allow the user or care provider the ability to retract the tubing at the push of a button. The apparatus would be provided with a hand grip and belt clip to enhance handling and portability. As such, the patient would no longer be encumbered by excessive slack oxygen tubing when moving between locations.

### RELATED APPLICATIONS

The present invention was first described on May 17, 2007 in a notarized, Official Record of Invention on file at the Offices of Invent SAI in Washington, Pa. There are presently no filed, nor currently any co-pending applications, anywhere in the world.

### I. FIELD OF THE INVENTION

The present invention relates generally to an oxygen tubing reel and, more particularly, to a convenient oxygen tubing reel capable of attachment to residential supplemental medical oxygen generating devices.

### II. MOTIVATION FOR THE INVENTION

Supplemental oxygen is common used by patients in hospitals and extended care facilities. These facilities use a central oxygen station which delivers medical grade oxygen to multiple rooms, surgery centers and the like. Supplemental oxygen is also used in the home environment. In residential situations, a small oxygen generator or even a large tank is provided in one room with a long hose system to reach various points in the house. The hose system poses a tripping hazard to the patient, who is frequently elderly and others. Additionally, the patient must constantly battle the hose to keep it from becoming tangled, knotted or becoming caught on furniture legs with movement. Accordingly, there is a need for a means by which the disadvantages of long oxygen lines in a residential environment can be reduced or eliminated. The development of the invention herein described fulfills this need.

An apparatus to aid in the holding and retention of oxygen tubing used in a home health care environment, comprising approximately fifty (50) feet of clear oxygen tubing wound upon a retractable reel. A spring-loaded tensioning system is provided for automatic deployment and retraction of the oxygen line. The spring-loaded system would allow the user or care provider the ability to extend or retract the tubing or cable by simply tugging on it. The invention would be provided with a universal mounting bracket suitable for use on any make of oxygen generator or even oxygen tank. As such, the patient would no longer be encumbered by excessive slack when moving about in any room in their home.

### III. DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a front view of a retractable oxygen tubing reel 10, according to a preferred embodiment of the present invention;

FIG. 2 is a rear view of a retractable oxygen tubing reel 10, according to a preferred embodiment of the present invention;

FIG. 3a is a side cut-away view of a retractable oxygen tubing reel 10 depicting a fully retracted tubing state, according to a preferred embodiment of the present invention; and,

FIG. 3b is a front cut-away view of a retractable oxygen tubing reel 10 depicting a partially deployed tubing state, according to a preferred embodiment of the present invention.

### DESCRIPTIVE KEY

10 retractable oxygen tubing reel
20 housing
21 fitting well
25 hand grip opening
27 belt clip
30 latch
32 hinge
35 latch appendage
40 tubing
41 peripheral tubing
45 stop fitting
50 supply fitting
60 release mechanism
61 spool
63 spool groove
65 spool flange
100 nasal cannula
110 supply tube

### IV. DESCRIPTION OF THE INVENTION

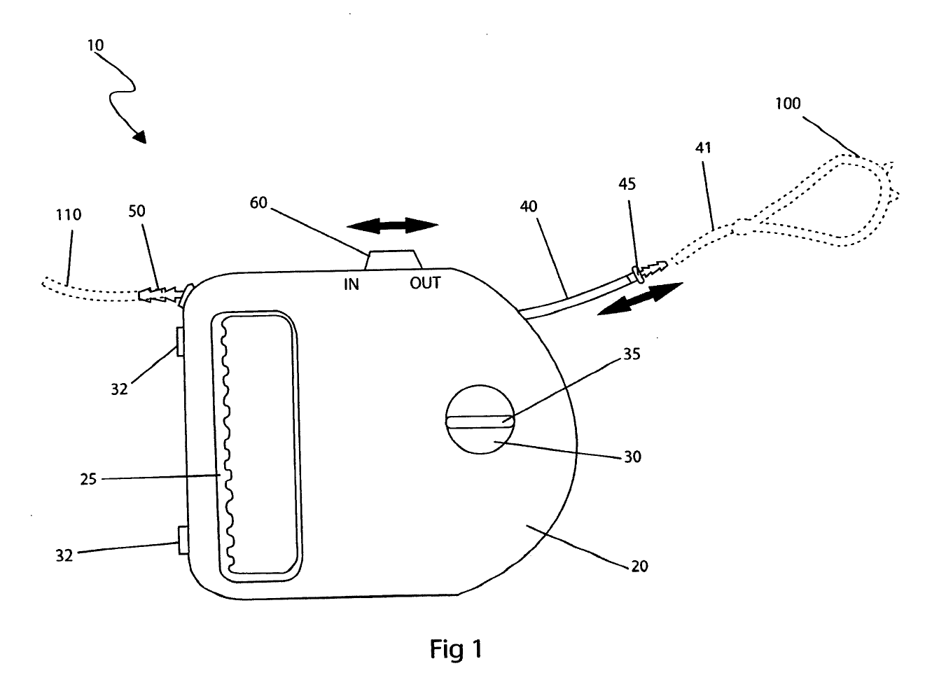

The best mode for carrying out the invention is presented in terms of its preferred embodiment, herein depicted within FIG. 1 through 3b.

### 1. Detailed Description of the Figure

The best mode for carrying out the invention is presented in terms of its preferred embodiment, herein depicted within FIGS. 1 through 3b. However, the invention is not limited to the described embodiment, and a person skilled in the art will appreciate that many other embodiments of the invention are possible without deviating from the basic concept of the invention and that any such work around will also fall under scope of this invention. It is envisioned that other styles and configurations of the present invention can be easily incorporated into the teachings of the present invention, and only one particular configuration shall be shown and described for purposes of clarity and disclosure and not by way of limitation of scope.

The terms “a” and “an” herein do not denote a limitation of quantity, but rather denote the presence of at least one of the referenced items.

The present invention describes an apparatus and method for a retractable oxygen tubing reel (herein described as the “apparatus”) 10, which provides an apparatus to aid in holding and retaining oxygen tubing 40 used in a home health care environment, comprising approximately fifty (50) feet of clear oxygen tubing 40. An internal spring-loaded tensioning spool 61 is provided for automatic retraction of the oxygen line 40. The spring-loaded system 61 would allow the user or care provider the ability to manually extend the tubing 40 and retract the tubing 40 using a release mechanism 60. The invention would be provided with a hand grip opening 25 and a belt clip 27 for convenient holding and transport. As such, a patient would no longer be encumbered by excessive slack tubing 40 when moving about in a hospital or in their home.

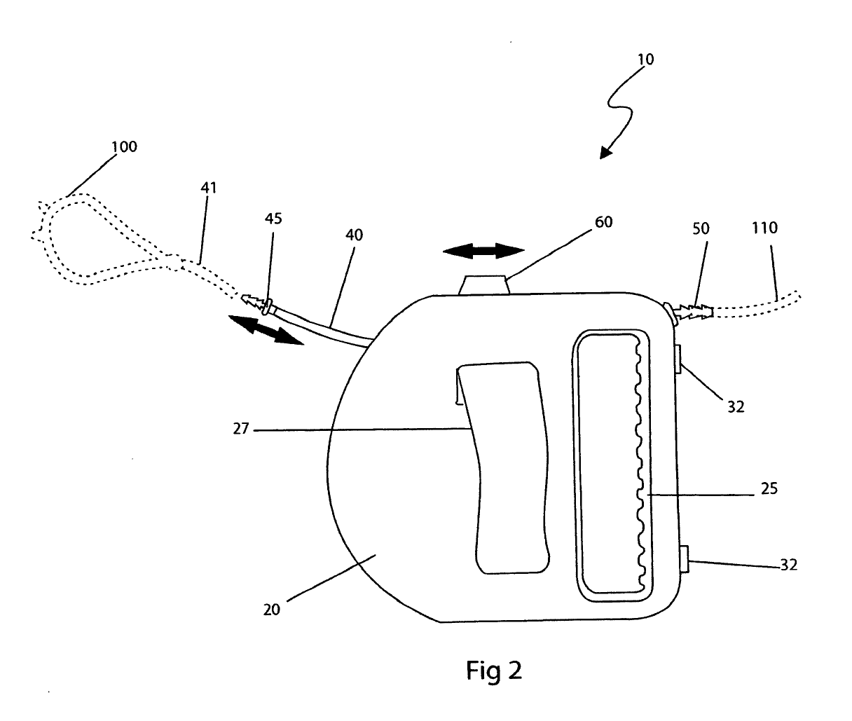

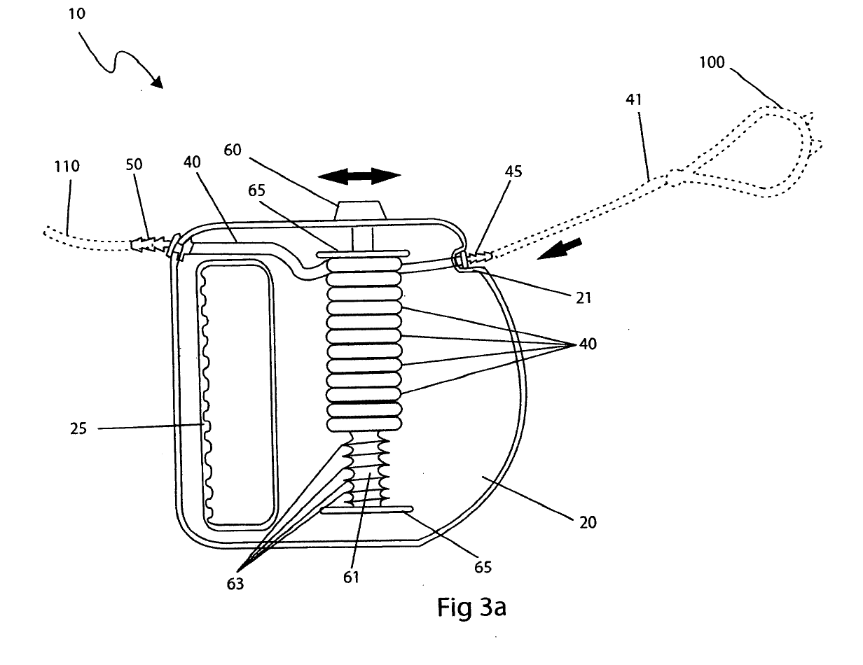

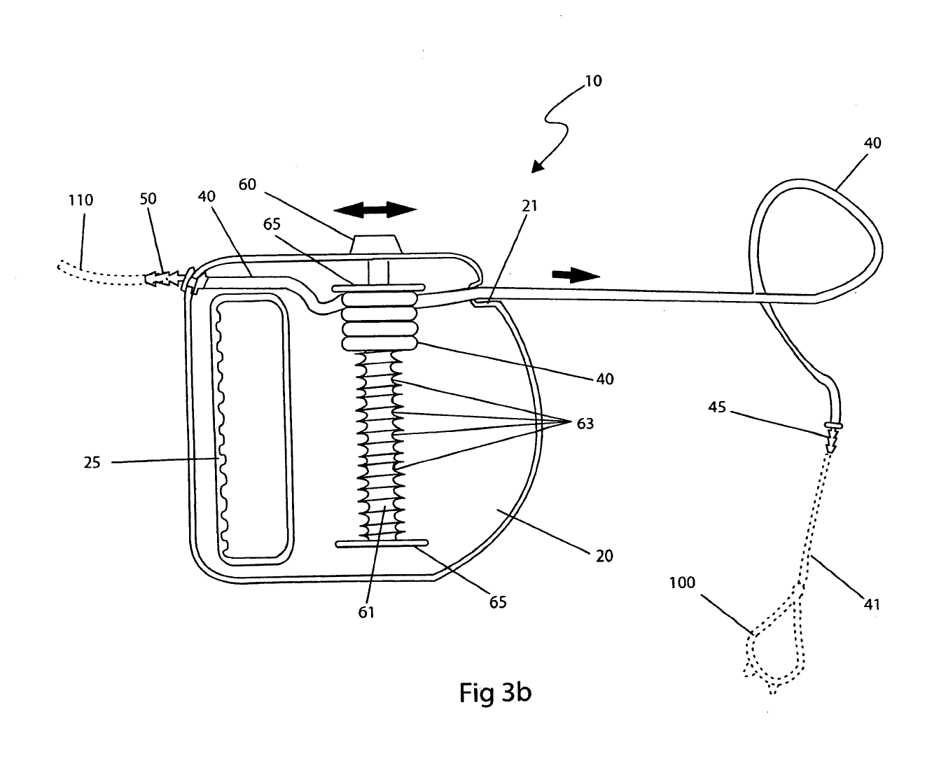

Referring now to FIGS. 1 and 2, a front view and a rear view of the apparatus 10, according to the preferred embodiment of the present invention, are disclosed. The apparatus 10 comprises a housing 20, a pair of hinges 32, and a latch 30. The housing 20 provides a compact and protective enclosure thereto a length of tubing 40 and internal tubing retracting devices (see FIG. 3a). The housing 20 comprises a generally rectangular enclosure being approximately six (6) to ten (10) inches square on a side and approximately two (2) inches thick and is envisioned being made of a durable rigid plastic materials produced in an injection molding process. The housing 20 is also envisioned being introduced in a variety of colors and patterns based upon a user's preference. The housing 20 further comprises user-friendly enhancements such as an internal hand grip opening 25 and a belt clip 27. The belt clip 27 may be integrally molded therein the housing 20 or affixed thereto a rear surface being fastened using rivets, screws, or the like. The belt clip 27 provides a hands-free method of carrying the apparatus 10 thereon one's person allowing a user to use both hands to perform various normal tasks while seated or standing. The hand grip opening 25 provides a gripping means thereto a user providing an oval-shaped aperture suitable for insertion of a user's four (4) fingers in an expected manner.

The housing 20 comprises a hollow two-piece hinged construction further comprising a pair of integral molded or metal hinges 32 and a front mounted latch 30. The latch 30 is located adjacent thereto the grip opening 25 providing an opening means thereto the housing 20 providing access thereto the oxygen tubing 40 for periodic cleaning or replacement as required. The latch 30 is envisioned to comprise a common cylindrical half-turn mechanical device providing a finger activated latch appendage 35 or similar grasping feature to gain access thereto an interior portion of the housing 20.

Referring now to FIG. 3a, a side cut-away view of the apparatus 10 depicting a fully retracted tubing state, according to the preferred embodiment of the present invention, is disclosed. The apparatus 10 as illustrated here depicts a retracted and stored length of oxygen tubing 40 wrapped therearound a spool 61. The oxygen tubing 40 further comprises a stop fitting 45 and a supply fitting 50. The oxygen tubing 40 is envisioned to provide a length of approximately fifty (50) feet with a connecting means thereupon both ends allowing secure and sealed attachment thereto peripheral devices and/or tubing via the stop fitting 45 and the supply fitting 50. The stop fitting 45 is envisioned to be permanently affixed thereto the extendable end of the oxygen tubing 40 using plastic welding, adhesives, or the like. The stop fitting 45 further provides an attachment means thereto peripheral tubing 41 and/or a nasal cannula 100 using common molded features such as barbs, annular friction features, or the like, providing a secure and sealed fit thereto peripheral tubular devices 41. The stop fitting 45 also comprises a molded-in annular ring feature providing an external retaining means thereto the oxygen tubing 40 when fully retracted. The stop fitting 45 is envisioned being held therewithin a female cylindrical fitting well 21 being integral thereto the housing 20. The supply fitting 50 is located along a hinged 32 end of the housing 20 and provides a permanent sealed connection thereto the oxygen tubing 40 in a similar manner as the stop fitting 45. The supply fitting 50 further provides a stationary connection thereto an oxygen supply tube 110 along an external surface of the housing 20 as shown. The supply fitting 50 comprises particular molded-in circular female features so as to provide an entrapment thereto an aperture feature integral thereto the housing 20.

The apparatus 10 further provides a means to store, extend, and retract the oxygen tubing 40 therefrom the housing 20 comprising a manual release mechanism 60, a spool 61, a plurality of spool grooves 63, and a pair of spool flanges 65. The manual release mechanism 60 provides automatic retraction of the oxygen tubing 40 therefrom am extended state thereto a retracted and stored state therewithin the housing 20 via a vertically mounted internal spool 61. The release mechanism 60 is envisioned to be a plastic push button, sliding switch, or the like, providing a mechanical locking function thereto the spool 61. The spool 61 is envisioned to comprise an internal friction device providing slight rotating resistance thereto the spool 61 during deployment of the oxygen tubing 40. Additionally, the spool 61 is envisioned to comprise an internal pre-loaded radial spring device which upon activation of the release mechanism 60, provides a torque thereto the spool 61 being sufficient so as to rotate said spool 61 and retract the oxygen tubing 40 therewithin the housing 20. The spool 61 is also envisioned to be made of a rugged metal or plastic material further comprising integral features to prevent tangling of said oxygen tubing 40 therewithin the housing 20 including a plurality of spool grooves 63 and a pair of spool flanges 65. The spool grooves 63 comprise a radial spiral pattern and a particular female diameter corresponding thereto a profile of the oxygen tubing 40, thereby propagating smooth reeling of the oxygen tubing 40 during automatic retraction thereof in an expected manner.

Referring now to FIG. 3b, a side cut-away view of the apparatus 10 depicting a partially deployed tubing state, according to the preferred embodiment of the present invention, is disclosed. The apparatus 10 is illustrated here depicting an in-use state having an extended length of oxygen tubing 40 being connected thereto a peripheral length of tubing 41 and a common nasal cannula 100.

It is envisioned that other styles and configurations of the present invention can be easily incorporated into the teachings of the present invention, and only one particular configuration shall be shown and described for purposes of clarity and disclosure and not by way of limitation of scope.

### 2. Operation of the Preferred Embodiment

The preferred embodiment of the present invention can be utilized by the common user in a simple and effortless manner with little or no training. After initial purchase or acquisition of the apparatus 10, it would be utilized as indicated in FIG. 1.

The method of utilizing the apparatus 10 may be achieved by performing the following steps: connecting the supply fitting 50 thereto an oxygen supply tube 110 using integral connecting features; connecting the stop fitting 45 thereto peripheral devices 41 such as a nasal cannula 100 using integral connecting features; deploying a desired length of oxygen tubing 40 therefrom the housing 20; applying said nasal cannula 100 thereto a patient's head and nose areas in a normal manner; initiating and regulating a supply of oxygen therefrom a supply source 110 thereto the apparatus 10; deploying an additional length of oxygen tubing 40 therefrom the housing 20 necessary to facilitate a laying, standing, or walking position; grasping the apparatus 10 during manipulation thereof during normal activities and/or tasks using the hand grip opening 25; clipping the apparatus 10 thereto ones belt or clothing using the belt clip 27, thereby providing greater freedom to use one's hands to perform various tasks; retracting all or a length of the oxygen tubing 40 thereinto the housing 20 by pressing the activation mechanism 60 as desired; opening the housing 20 using the latch 30 and hinges 32 for periodic cleaning or replacement of the oxygen tubing 40 as needed; and, benefiting from the portability of the apparatus 10 as well as the convenient retraction and storage of an excess length of oxygen tubing 40 during the application of oxygen thereto patients in a hospital and/or home setting.

An alternative method of use of the present invention 10 comprises using the apparatus 10 to store and administer excess intravenous or specialty gas tubing.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

1-3. (canceled)

4. A holder/reel that will release tubing on demand with patient activity then recoil excess tubing when patient is immobile.

5. The holder/reel described in claim 4 is a safety device designed to help prevent falls/injury to oxygen patients and caregivers by removing excess tubing from floors and walkways.

6. The holder/reel comprised of a lightweight housing with a soft hand grip; a securing device such as a belt clip for hands free mobility; a slide latch; a hinged opening; a release mechanism; a grooved spool to maintain integrity of supply tubing; a spool flange; a peripheral stop fitting; a tubing supply fitting; supply tubing not to exceed 50 feet; a housing bolt/screw; oxygen delivery device nasal cannula/mask.
